(19) United States
(12) Patent Application Publication
(10) Pub. No.: US 2017/0105947 A1
(43) Pub. Date: Apr. 20, 2017

(54) Appetite Suppressant Composition

(71) Applicant: Julio Lionel Pimentel, Buford, GA (US)

(72) Inventor: Julio Lionel Pimentel, Buford, GA (US)

(21) Appl. No.: 14/887,521

(22) Filed: Oct. 20, 2015

(51) Int. Cl. A61K 31/11; A61K 9/68

(52) U.S. Cl. CPC A61K 31/11; A61K 9/0058

(57) ABSTRACT

A method to control appetite and/or food intake in humans and companion animals by the use of an encapsulated six-carbon alpha, beta-unsaturated aldehydes. The preferred aldehyde is trans-2-hexenal.

### FIELD OF THE INVENTION

The present invention relates to a method to control appetite and reduce food intake in humans and companion animals by using non-encapsulated or encapsulated/protected trans-2-hexenal. In humans the invention is delivered as component of a chewing gum and in companion animals by spraying the invention in top of the extruded food.

### DISCUSSION OF THE BACKGROUND

Worldwide in 2014, 39% of people are overweight and 13% are obese. In the U.S., more than 65% of the population is overweight and one-third of adults are obese (34.9%, or 78.6 million). This overweight population has resulted in an increase of a variety of related health conditions, including heart disease, stroke, type 2 diabetes and certain types of cancer. The estimated annual medical cost of obesity in the U.S. was $147 billion in 2008. This is also happening in children between 2-19 years, where the prevalence of overweight children is 17%. Obesity has become a leading cause of preventable death (www.nutraceuticalsworld.com/issues/2015-09/view_features/delivering-effective-weight-management-solutions/?email_uid=88cb5c460d/list_id=396c189146/#sthash.x8E8FUA4.dpuf).

There has been an increase in obesity in countries where not long ago they had an ideal body weight; this is the case like China and Japan. People from these countries that have migrated to the U.S. are in their majority as heavier as their American counterparts.

The calorie-dense Western-type diet, super-sizing meals, overeating, snacking and sedentary lifestyle are considered the risk factors (www.nutraceuticalsworld.com/issues/2015-09/view features/the-key-to-healthy-weight-good-fat-in-excess-fat-out/?email_uid=88cb5c460d/list_id=396c189146/#sthash.VBNbnSiX.dpuf).

There are not secret weapon for decreasing body weight or appetite, all drugs, herbs and exercise programs work because they reduce food intake or our body expend more energy than the one ingested. All these procedures kept your body weight down as long as you keep in the program. Even thermogenic products work as long as they are taken frequently. Taking these types of products keep your body in a high metabolic rate where protein, instead of fat, is utilized as source of energy. Almost 100% of all dieters come back to their pre-program weight within two years.

Surgical operation such as stomach stapling is expensive and requires several hospitalization days. Liposuction takes out the fat but if the patient does not change his/her lifestyle, fat will accumulate in other areas.

Products like lipase inhibitors or other digestive enzymes (amylases, proteases) have been developed. The pancreatic lipase inhibitor Orlistat (tetrahydrolipstatin) has several known side effects, including unpleasant gastrointestinal issues-steatorrhea, fecal incontinence, frequent bowel movements and urgency, compromised absorption of fat-soluble vitamins and nutrients, increased appetite and diminished effectiveness over time (www.nutraceuticalsworld.com/issues/2015-09/view features/the-key-to-healthy-weight-good-fat-in-excess-fat-out/?email_uid=88cb5c460d/list_id=396c189146/#sthash.VBNbnSiX.dpuf). A couple patents (U.S. Pat. Nos. 7,618,624 and 7,334,713) using natural lipase inhibitors based on avian antibodies reported an in vitro inhibition of pancreatic lipase from 5 to 50% depending on the dosage. The addition of the anti-lipase antibody in drinking water resulted in the same feed intake than the untreated control but rats in the antibody-fed group had lower body weight gain. The antibody has no effect in amylase or proteases.

Body weight regulation products have secondary side effects and their effectiveness and mode of action varies. Commercial products like Xanthigen target pathways involved in body weight regulation including adipocyte differentiation, resting energy expenditure and lipid metabolism. Satiereal promotes satiety and helps avoid snacking and compulsive eating behaviors. Adipromin targets body composition by inhibiting adipose cell formation and enhancing the breakdown of fatty tissues (www.nutraceuticalsworld.com/issues/2015-09/view_features/delivering-effective-weight-management-solutions/?email_uid=88cb5c460d/list_id=396c189146/#sthash.x8E8F UA4.dpuf). WellTrim iG targets satiety, appetite control, thermogenesis, and blocks the conversion of blood sugar into stored fat. DygloFit promotes satiety, appetite control, thermogenesis and has antioxidant activity (www.nutraceuticalsworld.com/issues/2015-09/view_features/delivering-effective-weight-management-solutions/?email_uid=88cb5c460d/list_id=396c189146/#sthash.x8E8F UA4.dpuf).

Other methods to decrease food intake includes water preloading. In a recent study, Eighty-four obese adults were recruited having half of them consuming 500 ml of water 30 min before their main meals. After 12-week follow-up, the water preloading group lost 1.3 kg (P=0.028) more than the control group (Parretti, Helen M., et. al. 2015. Efficacy of water preloading before main meals as a strategy for weight loss in primary care patients with obesity. Obesity 23: (9) 1785-1791).

Intake of liposomes made of fractionated oat oil (LOO) affected the digestion process and postprandial lipemia and induced satiety. Intake of 14 to 35 g lipids from LOO significantly increased plasma concentrations of CCK, GLP-1, GLP-2, and PYY postprandially (Ohlsson, L. et. al. 2014. Postprandial effects on plasma lipids and satiety hormones from intake of liposomes made from fractionated oat oil: two randomized crossover studies. Food & Nutrition Research 58: 244-65).

Thylakoids, membranes in the chloroplasts of green leaves, have been shown to promote satiety and suppression of hunger. Administration of thylakoids in human effectively reduces body weight and improves glucose/lipid metabolism in overweight individuals. In animal studies thylakoids were mixed with food and in human studies thylakoids were mixed with food ingredients as part of a complete meal or served as a juice prior to a meal. Thylakoids slow down digestion of dietary fat in a way that promotes the release of CCK during a longer period of time (Erlanson-Albertsson and Per-Ake Albertsson, 2015. The Use of Green Leaf Membranes to Promote Appetite Control, Suppress Hedonic Hunger and Loose Body Weight Charlotte. Plant Foods Hum. Nutr. 70:281-290).

### SUMMARY OF THE INVENTION

The present invention relates to a method to control appetite and reduce food intake in humans and companion animals. In humans by supplying encapsulated/protected trans-2-hexenal as part of a chewing gum composition and in companion animals by spraying encapsulated/protected trans-2-hexenal in top of the extruded food. The gum, which is used prior to any meal or at any time , is composed by standard ingredients with the differences of the addition of acid-resistant, protease-resistant, amylase-resistant encapsulated trans-2-hexenal which increases CCK levels in the intestinal tract therefore suppressing appetite or decreasing food intake.

The present invention also relates to applying the encapsulated/protected trans-2-hexenal on top of companion animal food in order to decrease the amount of food intake. It is an object of the present invention to provide a natural mean to decrease control appetite or decrease food intake in humans.

It is an object of the present invention to provide a natural mean to decrease or limit food intake in companion animals.

An object of the invention is a flavoring agent composed of an encapsulated trans-2-hexenal.

The encapsulated t-2-hexenal is acid-resistant to prevent been absorbed in the stomach.

The encapsulated t-2-hexenal is amylase-resistant to prevent lingual and stomach amylase from degrading the encapsulate.

The encapsulated t-2-hexenal may be encapsulated by any other form in order to reach the intestinal tract without affecting the intent of the present invention.

The encapsulated t-2-hexenal is an ingredient for chewing gum.

The encapsulated t-2-hexenal may be replaced by other alpha-beta-unsaturated aliphatic aldehydes.

The encapsulated t-2-hexenal may be added as % of the gum formulation.

The encapsulated t-2-hexenal is effective in increasing CCK in the intestine.

The encapsulated t-2-hexenal is effective in decreasing food intake.

The encapsulated t-2-hexenal when reaching the small intestine lumen increases CCK level.

The encapsulated t-2-hexenal increases CCK level in the intestine lumen in a dose depending matter.

The encapsulated t-2-hexenal optimal level varies from 0.01 ppm to 50,000 ppm, preferably 10 to 1000 ppm.

The encapsulated t-2-hexenal can de apply orally by other means i.e. aerosol, propulsion spray, dropwise without affecting the intended use of the present invention.

The encapsulated t-2-hexenal can be applied on top of extruded companion animal food.

The encapsulated t-2-hexenal can be mixed with water, fat or palatants before applied to companion animal food.

The encapsulated trans-2-hexenal decreases food intake in companion animal food.

### DESCRIPTION OF THE PREFERRED EMBODIMENTS

This invention discloses a method to suppress appetite or decrease food intake by increasing CCK level (appetite suppressant hormone) by using an encapsulated/protected trans-2-hexenal in chewing gum or sprayed on food.

The preferred encapsulated alpha-beta-unsaturated aliphatic aldehyde of the invention is trans-2-hexenal but other can be used i.e. propenal, trans-2-butenal, 2-methyl-2-butenal, 2-methyl-(E)-2-butenal, 2-pentenal, trans-2-hexen-1-ol, 2-methyl-2-pentanal, 2-isopropylpropenal, 2-ethyl-2-butenal, 2-ethyl-2-hexenal, (Z)-3-hexenal, 3,7-dimethyl-6-octenal, 3,7-dimethyl-2,6-octadienal, (2E)-3,7-dimethyl-2-6-octadienal, (2Z)-3,7-dimethyl-2,6-octadienal, trans-2-nonenal, (2E,6Z)-nonadienal, 10-undecanal, 2-dodecenal and other alpha, beta.-unsaturated aliphatic aldehydes including trans, trans-2, 4-di-unsaturated aldehydes; trans, cis-2, 4-di-unsaturated aldehydes; cis, trans-2, 4-di-unsaturated aldehydes; and cis, cis-2, 4-di-unsaturated aldehydes with flavoring properties as well as their respective alcohol and acid forms.

Several patents suggest the use of trans-2-hexenal as a fragrance or perfume. U.S. Pat. No. 6,596,681 suggests using trans-2-hexenal as a fragrance in a wipe for surface cleaning. U.S. Pat. No. 6,387,866, U.S. Pat. No. 6,960,350 and U.S. Pat. No. 7,638,114 suggest using essential oils or terpenes (for example trans-2-hexenal) as a perfume for antimicrobial products. U.S. Pat. No. 6,479,044 discloses an antibacterial solution comprising an anionic surfactant, a polycationic antibacterial and water, where an essential oil is added as a perfume. This perfume could be a terpene such as trans-2-hexenal or another type of terpene. U.S. Pat. No. 6,323,171, U.S. Pat. No. 6,121,224 and U.S. Pat. No. 5,911,915 disclose antimicrobial purpose microemulsions containing a cationic surfactant where an essential oil is added as a perfume. This perfume can be various terpenes including trans-2-hexenal. U.S. Pat. No. 6,960,350 discloses an antifungal fragrance where a synergistic effect was found when different terpenes were used in combination (for example trans-2-hexenal with benzaldehyde).

None of the patents or literature shown above described any effect of the encapsulated unsaturated aldehydes on appetite control or food intake.

A new US patent application, “Cholecystokinin Secretion-Promoting Composition” (US Patent Application #20150164823), shows that trans, trans-2, 4-decadienal added to the lumen of the intestine increased the level of CCK in vitro. This cholecystokinin secretion-promoting composition contains an unsaturated aldehyde having a main chain of 4-12 carbon atoms having a double bond in at least position 2 or 4, wherein the main chain has 4-9 carbon atoms. This cholecystokinin secretion-promoting composition can be used as an appetite suppressant. By adding this cholecystokinin secretion-promoting composition to food, appetite-suppressing food products can be provided.

Oral administration of 50-100 mg/kg of decadienal in rats reduced gastric emptying possible through stimulation of the serotonin-producing entero-endocrine cells (Tira, T. et. al. 2015. A di-unsaturated aldehyde; trans, trans-2, 4-decadienal in the intestinal lumen suppresses gastric emptying through serotonin signaling in rats. J. Agri. Food Chem. Web publication date: Aug. 31, 2015).

This US patent application (#20150164823) and the paper cited above failed to mention the use encapsulated/protected trans-2-hexenal.

U.S. Pat. No. 4,241,098, described is a method for augmenting or enhancing the aroma or flavor of a foodstuff comprising adding to the foodstuff from 0.05 up to about 500 parts per million of a mixture of cis-3-hexenal, trans-2-hexenal, cis-3-hexenyl formate. This invention to be used in foodstuff flavors, chewing gum flavors, toothpaste flavors and medicinal product flavors, to augment or enhance the powerful green, leafy, fruity, fatty and spicy aromas.

The present invention includes C3 to C12 alpha-beta-unsaturated aliphatic aldehydes, preferably trans-2-hexenal.

By using the present invention, the amount of CCK is increased which has been shown that has appetite suppressant activity.

### Definitions

A “volume percent” of a component is based on the total volume of the formulation or composition in which the component is included.

By the term “effective amount” of the encapsulated t-2-hexenal is meant such amount capable of performing the function of the compound or property for which an effective amount is expressed, such as a non-toxic but sufficient amount to provide the desired appetite suppressant or decreased food intake effect. Thus an appropriate effective amount may be determined by one of ordinary skill in the art using only routine experimentation.

The encapsulated t-2-hexenal percentage used in the gum formulation can vary depending on the ingredients of the chewing gum and the effective amount of the encapsulated t-2-hexenal used.

Encapsulated or protected trans-2-hexenal also mean are used interchangeably in this application meaning both that the aldehyde is resistance to many physiological or environmental changes before reaching the gastro-intestinal tract. The words encapsulated and protected are interchangeable meaning that both refer to improve stability of trans-2-hexenal so it can reach the intestine where it is released.

### Compositions

The volatile compound used in this invention is trans-2-hexenal, which is six carbon, double bond aldehyde, C₆H₁₀O and MW=98.14. Natural or synthetic trans-2-hexenal, also known as leaf aldehyde is considered a natural flavoring according to EU Council Directive 88/388/EEC and US FDA 21CFR101.22.

It is not a scope of this invention to have a new chewing gum formulation but an encapsulated additive to the current commercially available chewing gums. For better comprehension of the invention an explanation of what encapsulation is follows. Encapsulation delays the release of the active substance from the gum composition. The methods of encapsulating the active ingredient/flavor include, but are not limited to, agglomeration, spray drying, spray congealing, fluid bed coating, coacervation, fiber spinning and combinations thereof. Encapsulation material can include inulin, fructooligosharides, trehalose, mannose, b-glucans and other carbohydrates, synthetic encapsulates, fat based encapsulate. Any encapsulating material would be satisfactory that delays the release of the active ingredient (U.S. Pat. No. 5,139,794 A).

Chewing gums containing gum base, sweetening and bulking agents, a flavoring agent, and flavor encapsulated in a manner to delay its release from the gum composition. A chewing gum composition typically comprises a water-soluble bulk portion, a water-insoluble chewable gum base portion and typically water-insoluble agents. The water-soluble portion dissipates with a portion of the flavoring agent over a period of time during chewing. The gum base portion is retained in the mouth throughout the chew. The insoluble gum base generally comprises elastomers, resins, fats and oils, waxes, softeners and inorganic fillers. The water-soluble portion of the chewing gum contains a bulking and sweetening agent. In most chewing gums, the sweetening agent is sucrose or sugar substitutes. The flavoring agent used in the gum may be present in an amount within the range of from about 0.001 to about 10.0 weight percent and preferably from about 0.05 to about 3.0 weight percent of the gum. The flavoring agents may comprise essential oils, synthetic flavors, or mixture thereof including, but not limited to, oils derived from plants and fruits such as citrus oils, fruit essences, peppermint oil, spearmint oil, clove oil, oil of wintergreen, anise, and the like.

The active substances formulated within the chewing gum are released by chewing and been moistened with saliva. These substances then are swallowed with saliva and entered into the gastrointestinal track. These substances are either a soluble or a suspension, which in both cases has more rates of absorption than other forms (e.g., tablets). The possibility of controlling the active ingredient release over an extended time is an advantage of medicated chewing gums (Aslani and Jalilian, 2013. Design, formulation and evaluation of caffeine chewing gum. Adv. Biomed Res. 2013; 2: 72).

Chewing forces releases the volatile flavor compounds into the exhaling air to the nasal compartment. Flavor releases from chewing gum is governed by chewing frequency (CF), masseter muscle activity (MMA), chewing force (CFO), and saliva flow rate (SFR). More than 50% of the released menthol and menthone could be retrieved in the expired air and saliva. After 2-min of chewing, the concentration of flavor compounds in the expired air depended primarily on MMA and CF, becoming higher with increased MMA and CF. The concentration of flavor compounds in saliva depended primarily on SFR and the duration of the chewing task, becoming lower with high SFR and prolonged chewing duration. An increased volume of saliva in the mouth keeps more flavor compounds in the aqueous phase, thereby diminishing the release via the retronasal route (Haahr AM, et. al. 2004. Release of peppermint flavor compounds from chewing gum: effect of oral functions. Physiol. Behay. 82(2-3):531-40).

U.S. Pat. No. 4,241,098, described is a method for augmenting or enhancing the aroma or flavor of a foodstuff comprising adding to the foodstuff from 0.05 up to about 500 parts per million of a mixture of cis-3-hexenal, trans-2-hexenal, cis-3-hexenyl formate. This invention to be used in foodstuff flavors, chewing gum flavors, toothpaste flavors and medicinal product flavors, to augment or enhance the powerful green, leafy, fruity, fatty and spicy aromas. This patent fails to show an effect of an encapsulated aldehyde on appetite or food intake.

Humans are exposed daily to trans-2-hexenal through consumption of food and beverages. Human exposure to trans-2-hexenal is about 350 ug/kg/day, with 98% derived from natural sources and 2% from artificial flavoring. It is unlikely for trans-2-hexenal to be toxic to humans since toxic levels in rats are 30 times higher than the normal intake by humans (Stout, M. D. et. al. 2008. Toxicity, DNA Binding, and Cell Proliferation in Male F344 Rats following Short-term Gavage Exposures to Trans-2-Hexenal. Soc. Toxicological pathology (24):1533-1601).

Frying and cooking of polyunsaturated fatty acid (PUFA)-rich culinary oils generates high levels of cytotoxic aldehydic products, predominantly trans-2-alkenals, trans,trans-alka-2,4-dienals, cis,trans-alka-2,4-dienals, and n-alkanals, species arising from the fragmentation of conjugated hydroperoxydiene precursors. All are readily absorbed from the gut into the systemic circulation in vivo, metabolized and then excreted in the urine as C-3 mercapturate conjugates in rats (Grootveld, M. et. al. 1998. In Vivo Absorption, Metabolism, and Urinary Excretion of a,b-unsaturated Aldehydes in Experimental Animals Relevance to the Development of Cardiovascular Diseases by the Dietary Ingestion of Thermally Stressed Polyunsaturated-rich Culinary Oils. J. Clin. Invest. (101) 6:1210-1218).

In a rat study, feeding trans-2-hexenal at dietary levels of 0, 260, 640, 1600 or 4000 ppm fed for 13 wk. did not induce any changes in hematological parameters or organ weights. At 4000 ppm there was a no significant reduction in body weight and intake (Gaunt, I. F. and Colley, J. 1971. Acute and Short-term Toxicity Studies on trans-2-Hexenal. Fd Cosmet. Toxicol. (9): 775-786).

The previous study shows that high levels of trans-2-hexenal did not significantly affect feed intake or body weight, this study did not take into account the volatility, stability or other environmental factor that may have contribute to the stability of trans-2-hexenal in feed.

Several patents suggest the use trans-2-hexenal as a perfume/fragrance (U.S. Pat. No. 6,596,681, U.S. Pat. No. 6,387,866, U.S. Pat. No. 6,323,171, U.S. Pat. No. 6,121,224, U.S. Pat. No. 6,960,350 and U.S. Pat. No. 7,638,114), as antimicrobial (U.S. Pat. No. 6,479,044, U.S. Pat. No. 5,911,915, U.S. Pat. No. 6,960,350), but they all failed to show an effect on appetite or food intake.

The new US patent application, Cholecystokinin Secretion-Promoting Composition (US Patent Application #20150164823), shows that trans, trans-2, 4-decadienal added to rat intestinal cells in vitro increased the level of CCK and can be used as an appetite suppressant. By adding this cholecystokinin secretion-promoting composition to food, appetite-suppressing food products can be provided. This US patent application failed to mention the use of trans-2-hexenal. Also it fails to shown the effect of volatility of the aldehyde since the majority of these flavoring agents are not stable at room temperature. This application also fails on using the encapsulated aldehyde to decrease food intake in humans and companion animals.

Oral administration of 50-100 mg/kg of decadienal in rats reduced gastric emptying possible through stimulation of the serotonin-producing entero-endocrine cells (Tira, T. et. al. 2015. A di-unsaturated aldehyde, trans, trans-2, 4-decadienal in the intestinal lumen suppresses gastric emptying through serotonin signaling in rats. J. Agri. Food Chem. Web publication date: Aug. 31, 2015). As above this report also fails to disclose the stability of the aldehyde and its use in encapsulated form for humans and companion animals.

It is of the inventor experience that a product containing high level of trans-2-hexenal (32,000 to 40,000 ppm/kg feed) when fed to chickens, it decreased body weight and feed intake. That difference was not due to toxicity but other type of signal, presumably CCK. During this experience non-encapsulated trans-2-hexenal in combination with other chemicals were applied to feed. This experience did not take into account the stability of the aldehyde and the scope of that study was not as this invention suggest. In another personal observation, dogs preferred the untreated control than food containing 2,500 ppm of non-encapsulated trans-2-hexenal. As above, this experience did not take into account the stability of the aldehyde and the scope of that study was not as this invention suggest.

With the material shown above, the inventor suggests the use of encapsulated trans-2-hexenal to control appetite and decrease food intake in humans and companion animals.

Throughout this disclosure various publications and personal observations are referenced, which are all hereby incorporated by reference in their entireties into this application. It will be apparent for those skilled in the art that a number of modifications and variations may be made without departing from the scope of the present invention as set forth in the appending claims.

What I claim is:

1. A method for controlling appetite and decreasing food intake and appetite in humans and companion animals.

2. The method of claim 1, wherein an encapsulated trans-2-hexenal is given to human and companion animals.

3. The method of claim 1, wherein decrease of food intake or appetite control in humans is by mean of a chewing gum that contains encapsulated trans-2-hexenal.

4. The method of claim 1, wherein decrease of food intake in companion animals is by top dressing or spraying the encapsulated trans-2-hexenal to the companion animal food.

5. The method of claim 2, wherein the encapsulate delivers non-encapsulated trans-2-hexenal to the intestine where it is more active.

6. The method of claim 2, wherein the chewing gum comprising of encapsulated trans-2-hexenal increases the level of CCK in the gastrointestinal tract of humans.

7. The method of claim 2, wherein the encapsulated trans-2-hexenal increases the level of CCK in the gastrointestinal tract of companion animals.

8. The method of claim 6, wherein CCK controls appetite in humans.

9. The method of claim 6, wherein CCK decreases food intake in humans.

10. The method of claim 6, wherein the chewing gum is used as to control appetite at any time of the day.

11. The method of claim 6, wherein the chewing gum is used as to decrease food intake at any time of the day.

12. The method of claim 6, wherein level of trans-2-hexenal in the chewing gum varies from 0.0001% (1 ppm) to 5% (50,000 ppm), preferably 10 ppm to 1000 ppm.

13. The method of claim 2, wherein the increase on CCK by the chewing gum can also results from the consumption of other encapsulated aliphatic alpha, beta-unsaturated aldehydes.

14. The method of claim 7, wherein CCK controls appetite in companion animals.

15. The method of claim 7, wherein CCK decreases food intake in companion animals.

16. The method of claim 4, wherein controlling food intake in companion animals results from spraying encapsulated trans-2-hexenal on top of the mixed or extruded companion animal food.

17. The method of claim 2, wherein level of trans-2-hexenal applied to animal companion food varies from 0.001% (10 ppm) to 0.25% (2,500 ppm), preferably 100 ppm to 1000 ppm.

18. The method of claim 17, wherein the encapsulated or protected trans-2-hexenal is applied mixed with fat and palatants during the process of companion animal food manufacturing.

19. The method of claim 2, wherein the increase on CCK on companion animals can also results from the consumption of other encapsulated aliphatic alpha, beta-unsaturated aldehydes.
